(19) United States
(12) Patent Application Publication
(10) Pub. No.: US 2026/0115486 A1
(43) Pub. Date: Apr. 30, 2026

(54) IMPLANTABLE STIMULATION SYSTEM

(71) Applicant: AXONICS, INC., Irvine, CA (US)

(72) Inventors: Guangqiang Jiang, Irvine, CA (US); Xuechen Huang, Irvine, CA (US); Chun-Fu Chen, Palo Alto, CA (US); Faizal A. Sainal Abdeen, Mission Viejo, CA (US); Shanie E. Scoles, Mission Viejo, CA (US)

(73) Assignee: AXONICS, INC., Irvine, CA (US)

(21) Appl. No.: 19/368,645

(22) Filed: Oct. 24, 2025

Related U.S. Application Data
(60) Provisional application No. 63/713,524, filed on Oct. 29, 2024.

(51) Int. Cl. A61N 1/375; A61N 1/05; A61N 1/36; A61N 1/378

(52) U.S. Cl. CPC A61N 1/3756; A61N 1/0551; A61N 1/36007; A61N 1/3605; A61N 1/378

(57) ABSTRACT

A leadless neurostimulation device configured for implantation in a leg of a patient comprising a header with a first electrode and an elongated main body containing stimulation circuitry and a battery with a second electrode extending circumferentially around at least a portion of the main body. The electrode configuration proves greater volume of tissue activation with lower power requirements and improved battery efficiency.

### CROSS REFERENCE TO RELATED APPLICATIONS

The present application claims the benefit of and priority to U.S. Provisional Patent Application No. 63/713,524, titled IMPLANTABLE STIMULATION SYSTEM, filed Oct. 29, 2024, the disclosure of which is incorporated herein by reference.

### BACKGROUND

This application discloses a nerve stimulation system including an implantable pulse generator (IPG) for stimulating the tibial nerve.

Percutaneous tibial nerve stimulation (PTNS) is a clinically recognized treatment for overactive bladder (OAB) and its associated symptoms. The treatment involves periodic sessions of electrical stimulation delivered via a needle inserted near the ankle. While effective, PTNS requires ongoing monthly maintenance treatments, leading to a significant patient burden and high dropout rates due to the need for repeated needle insertions and associated discomfort. Studies indicate that a significant percentage of patients discontinue PTNS maintenance therapy after one year.

Implantable tibial nerve stimulation (ITNS) devices offer a potential solution to the limitations of PTNS by providing continuous stimulation, potentially improving patient adherence and treatment outcomes. However, current ITNS technologies face several challenges. These include the requirement for invasive implantation procedures for an IPG that includes long leads prone to fracture or migration, the need for multiple incisions or sutures to secure the IPG, reliance on external power sources hindering patient adherence, cumbersome external recharging systems, and limited device longevity due to small battery sizes. These limitations underscore the need for improved IPG device designs that prioritize patient comfort, minimize invasiveness, and ensure long-term efficacy.

### SUMMARY

Addressing these challenges, the current disclosure presents a novel implantable ITNS device designed as a fully self-contained unit. This innovative design integrates the electronics, primary battery, and electrodes into a single integrated device. Thus, the need for leads and the risks associated with their use and connection to a separate IPG. The ITNS device (e.g., an IPG) includes a titanium case or housing, a portion of which serves as the return electrode, and houses a hermetically-sealed electronics and a battery compartment. The device includes an epoxy header that incorporates a feedthrough element for an antenna. The antenna enables wireless programming for the device. The device also includes an electrode (e.g., functioning as a cathode) for delivering the nerve stimulation therapy.

The innovative integrated design of the ITNS device is configured to be implanted using a minimally invasive procedure that requires only a single skin incision. The size of the ITNS device is optimized for patient comfort and discretion. Also, the design of the integrated electrode provides for both effective stimulation of target nerve fibers and low electrode-tissue impedance. The size and configuration of the device maximizes battery efficiency, ensuring optimal therapeutic dosage and extending the operational life of the device.

In an example, a leadless neurostimulation device configured to be implanted in a leg of a patient has a header including a first electrode, an elongated main body containing circuitry for generating stimulation current and a battery, wherein the main body is connected to the header and includes a second electrode extending along a longitudinal section of the main body, and wherein the second electrode extends circumferentially around at least a portion of the main body.

Alternatively or additionally to any of the examples above, the neurostimulation device has the second electrode extending around the entire circumference of the main body.

Alternatively or additionally to any of the examples above, the neurostimulation device has the first electrode and the second electrode on the same side of the main body.

Alternatively or additionally to any of the examples above, the neurostimulation device has the first electrode including a curved surface to direct stimulation current for both deep and lateral neural stimulation.

Alternatively or additionally to any of the examples above, the header and elongated main body have a length, a width, and a thickness, the length exceeding the width, and the width exceeding the thickness, wherein the first electrode extends from a first lateral surface of the header and curves to an end surface of the header without crossing a midline of the thickness of the leadless neuromodulation device.

Alternatively or additionally to any of the examples above, the neurostimulation device has the first electrode as a cathode and the second electrode as an anode.

Alternatively or additionally to any of the examples above, the neurostimulation device has the battery as non-rechargeable.

Alternatively or additionally to any of the examples above, the neurostimulation device has the elongated main body including a non-conductive substrate with conductive regions defining the first and second electrodes.

Alternatively or additionally to any of the examples above, the neurostimulation device has the first and second electrode separated by a distance ranging from about 10 mm to 16 mm.

Alternatively or additionally to any of the examples above, the neurostimulation device has the first electrode with a surface area of approximately 30 mm² to 40 mm².

Alternatively or additionally to any of the examples above, the neurostimulation device has the second electrode with a surface area of about 260 mm² to 300 mm².

Alternatively or additionally to any of the examples above, the neurostimulation device exhibits an impedance of approximately 1 kΩ during operation.

Alternatively or additionally to any of the examples above, the neurostimulation device has the elongated main body including a housing including a sealed electronics compartment and a compartment for the battery, and the header includes a header portion connected to the housing.

Alternatively or additionally to any of the examples above, the neurostimulation device is configured to stimulate the tibial nerve.

Alternatively or additionally to any of the examples above, the neurostimulation device is configured to be implanted at a location about 2 cm to 8 cm above a medial malleolus of the patient.

In an example, a leadless neurostimulation device configured to be implanted in a leg of a patient has a header including a first electrode, an elongated main body containing circuitry for generating stimulation current and a battery, wherein the main body is connected to the header and includes a second electrode extending along a longitudinal section of the main body, and wherein the second electrode extends circumferentially around the majority of the main body.

Alternatively or additionally to any of the examples above, the neurostimulation device has the second electrode extending around the entire circumference of the main body.

Alternatively or additionally to any of the examples above, the neurostimulation device has the first electrode and the electrode on the same side of the main body.

Alternatively or additionally to any of the examples above, the neurostimulation device is configured to be implanted at a location about 2 cm to 8 cm above a medial malleolus of the patient.

Alternatively or additionally to any of the examples above, the neurostimulation device is configured to stimulate the tibial nerve.

Alternatively or additionally to any of the examples above, the neurostimulation device has the elongated main body including a non-conductive substrate with conductive regions defining the first and second electrodes.

In an example, an implantable pulse generator for providing stimulation pulses to a patient is configured to be implanted in the leg of the patient and has a header portion, a main body portion forming a case for stimulation circuitry and an energy storage device, a first electrode positioned on an exterior of the header portion, and a second electrode positioned on an exterior of the case.

Alternatively or additionally to any of the examples above, the implantable pulse generator has the first electrode and the second electrode on the same side of the implantable pulse generator.

Alternatively or additionally to any of the examples above, the implantable pulse generator has the second electrode wrapping completely around the case.

Alternatively or additionally to any of the examples above, the implantable pulse generator has the first electrode including a curved surface to direct stimulation current for both deep and lateral neural stimulation.

Alternatively or additionally to any of the examples above, the implantable pulse generator has the first and second electrode separated by a distance of about 10 mm.

Alternatively or additionally to any of the examples above, the implantable pulse generator lacks separate leads, lead connectors, and external lead attachment points.

In an example, an implantable pulse generator for stimulation of the tibial nerve has a housing including a sealed electronics compartment and a compartment for a battery, a header portion connected to the housing, a first electrode positioned on the header portion, and a second electrode extending along the housing and at least partially circumscribing the housing.

Alternatively or additionally to any of the examples above, the implantable pulse generator has the second electrode completely circumscribing the housing.

Alternatively or additionally to any of the examples above, the implantable pulse generator has the battery as non-rechargeable.

Alternatively or additionally to any of the examples above, the implantable pulse generator lacks separate leads, lead connectors, and external lead attachment points.

Alternatively or additionally to any of the examples above, the implantable pulse generator has the first electrode as a cathode and the second electrode as an anode.

Alternatively or additionally to any of the examples above, the implantable pulse generator has the first electrode and the second electrode separated by a distance ranging from 10 mm to 16 mm.

Alternatively or additionally to any of the examples above, the implantable pulse generator has the first electrode with a surface area of approximately 30 mm² to 40 mm².

The disclosed IPG provides a unique and improved volume of tissue activated (VTA) compared to the competition. VTA refers to the spatial spread of direct neural activation in response to electrical stimulation of the three-dimensional region of neural tissue that is directly influenced by the electrical field generated by a stimulating electrode. In general, VTA refers to the volume where the neurons are actually activated by the implanted device.

Further areas of applicability of the disclosed stimulation system will become apparent from the detailed description provided hereinafter. It should be understood that the disclosed detailed description and specific examples, while disclosing various embodiments, are intended for purposes of illustration only and are not intended to necessarily limit the scope of the disclosure.

### BRIEF DESCRIPTION OF THE DRAWINGS

FIG. 1A shows an exemplary ITNS system.

FIG. 1B shows an exploded ITNS system shown in FIG. 1A.

FIG. 2 shows an implant position for an ITNS system.

FIGS. 3-9 show exemplary IPG devices for ITNS according to several embodiments.

FIG. 10 shows the VTA of the various embodiments of disclosed IPGs.

FIG. 11 shows the VTA of a prior art system.

FIGS. 12A-12B show comparative spatial voltage distributions of a conventional device and a disclosed device.

FIGS. 13A-13B are comparative graphs between a conventional device and a disclosed device.

### DETAILED DESCRIPTION

A neural stimulation system may be used to apply one or more electrical impulses to targeted nerves, such as a tibial nerve, for treating disorders, including, but not limited to urological disorders. An implantable tibial nerve stimulation (ITNS) device may be utilized to deliver a therapy by stimulating a tibial nerve.

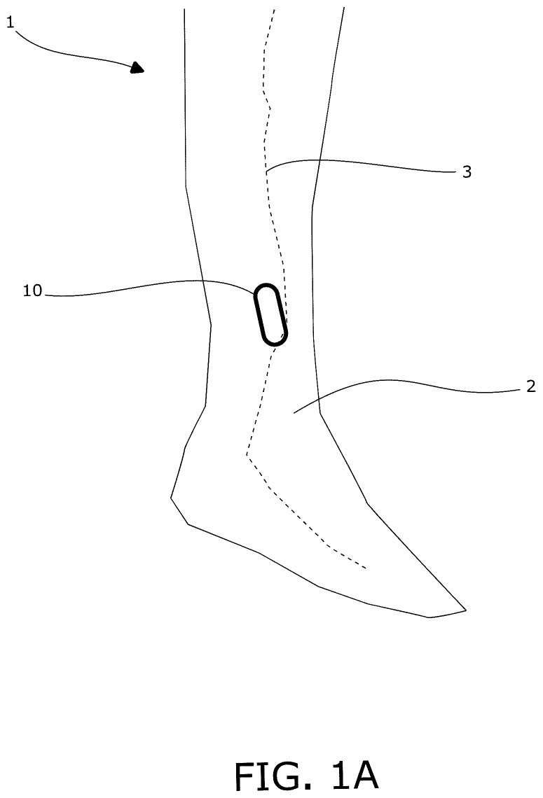

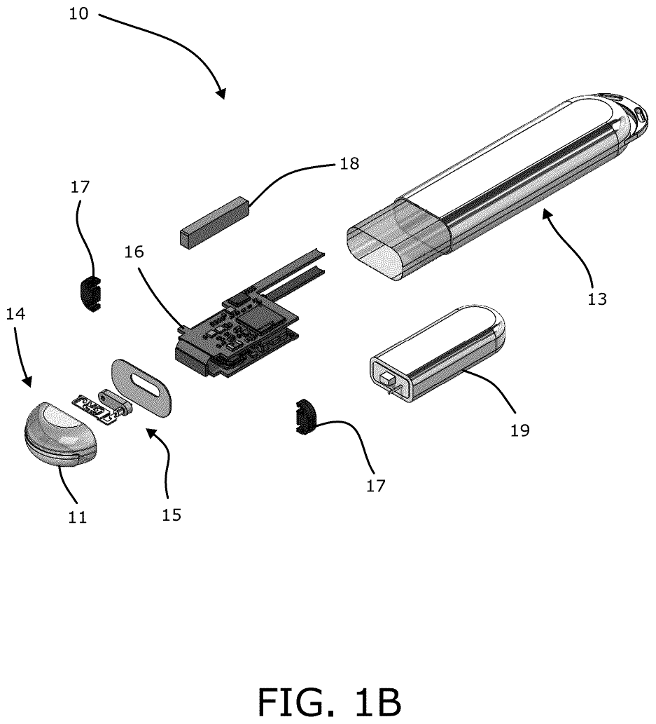

The typical location where an IPG 10 is implanted for ITNS is shown in FIG. 1A. According to one embodiment, the IPG 10 is preferably located 2 to 8 cm above a medial malleolus 2 of the patient 1. This location provides for sufficient tissue above the fascia to accommodate the device placement while ensuring the stimulation current reaches the tibial nerve 3 effectively.

FIG. 1B shows an exemplary embodiment of the IPG 10. In contrast to some conventional designs that require leads for an ITNS device to deliver the therapy, such leads and other lead attachment points (e.g., a lead connector) are notably absent in IPG 10. The IPG 10 is designed as a fully self-contained unit that integrates the electronics, primary battery, and electrodes into a single device. In some embodiments, the IPG 10 includes a non-rechargeable battery 19. As an additional benefit of the self-contained device, the procedure to implant the IPG 10 may be done with a single incision. In alternative examples, the non-rechargeable battery may be replaced by a rechargeable battery or a capacitor, and used in conjunction with a charging circuit to allow occasional or periodic recharging of the rechargeable battery or capacitor.

A header portion 14 and a main body 13 may be connected together to form the IPG 10. The main body 13 may form a partial enclosure, and may be formed, for example, by deep drawing and/or by welding or otherwise securing two or more pieces together. As shown, the main body 13 may include, optionally, one or more loops or openings for allowing a suture to be passed therethrough during implantation. Such sutures may be used to secure the device to the fascia. The suture openings may be omitted.

The IPG 10 may include the header portion 14 having a first electrode 11, which may be used as the cathode during therapy. When delivering therapy using first and second electrodes, the output may be a pulsed electrical signal, and may be monophasic, biphasic, or multiphasic. A biphasic pulse is commonly used for implantable devices, and includes a first phase having a first polarity and a second phase having a polarity opposite that of the first phase. As used herein, the “cathode” among a pair of electrodes is the electrode that is used as a cathode for the first phase of any biphasic or multiphasic output pulse(s), or it may be the electrode that is used as cathode during the only output forming a monophasic therapy pulse. The anode is then the electrode used as an anode during the first phase of a biphasic or multiphasic output and/or as the anode during the sole output of a monophasic therapy pulse.

The first electrode 11 forms a curved shape, having benefits as further detailed below. The first electrode 11 thus extends from a first longitudinal side of the housing and curves to form a portion of the end of the header/housing as well. The first electrode 11, does not, in this example, cross a midline of the device. The “midline” is the plane that bisects the device 10 to form the largest cross section thereof. Here, it can be seen that the device has a length in its longest dimension, a width in its next longest dimension, and a thickness in its shortest dimension. Midline, in reference to the electrode 11, is the plane on which the maximum length and width intersect. Thus the electrode does not cross the midline of the device in this example, but it does curve from the side surface parallel to the midline, to the end surface, which it only partly covers. Because the device will be implanted with one side down, it may be desirable to avoid allowing the electrode 11 to cross the midline. In other examples, the electrode 11 may extend fully around the end of the device and/or the device header. In an example, the electrode 11 is a thin shell that can be formed by cutting and stamping to shape, for example. In another example, the electrode 11 may be a machined part, formed by removal of material starting from a solid rectangle (or other shape).

A main body 13 of the IPG 10 may include a second electrode 12 (anode). The main body 13 (housing 13) may be formed of any suitable material; often metal is used for a conductive housing, for example, titanium, and/or stainless steel. A battery 19 and the printed circuit board and desiccant 18 may be housed in the body 13. The header may be separated by a feedthrough or sealing structure, and has the electrode 11 and also communications circuit and/or antenna 15. The header 14 is attached to form a hermetic seal for the device 10.

The PCB 16 may include any suitable electronics. Stimulation circuitry, a microcontroller, microprocessor, logic, or state machine, for example, may be positioned on the PCB. This may include sensing circuitry and/or output control circuitry. Sensing may be used to monitor a biological state of the patient and/or to monitor output of the device to measure, for example, impedance encountered by the output pulses. If a controller is included, it may execute stored instructions on a memory that may be included on the PCB 16. Any suitable arrangement of additional systems and circuitry may be included, such as additional logic, communications bus, application specific integrated circuits, etc. The memory may include any of RAM, ROM, and/or Flash memory, or other memory devices/media, and stores machine readable instructions for performing the methods disclosed herein and providing device configurations as described herein.

The stimulation circuitry issues therapy pulses, which may be directed in accordance with input/output circuitry (which may include protection circuitry for the device to avoid deleterious effects of MRI or other large magnetic or electrical fields). A communications circuit may be included, having a resonator, modulator, amplifier and/or antenna as needed by the particular implementation, and may come as a discrete chip. Commercially available chips for MedRadio and/or Bluetooth (including Bluetooth Low Energy) can be used, for example. The antenna 15 may be located in the header 14, if desired, to limit signal attenuation due to the housing 13. The communications circuit provides an interface for the device to communicate with external devices such as a clinician programmer and/or patient remote control, either of which may be used to control device settings (on/off) and/or program therapy parameters such as pulse amplitude, pulse width, pulse repetition rate, burst characteristics, quiescent periods between bursts, duty cycle and/or on/off periods, scheduled therapy, therapy ramp and/or turn-on/off characteristics, mono, bi, or multiphasic pulse configuration, pulse shape, polarity, or any other parameter that the skilled person may wish to control. External device communication can also be used to determine device status, including state of charge for the battery, presence of any errors in the device, and history of patient usage. Optionally the device may include other features such as an accelerometer which can be used to monitor patient activity.

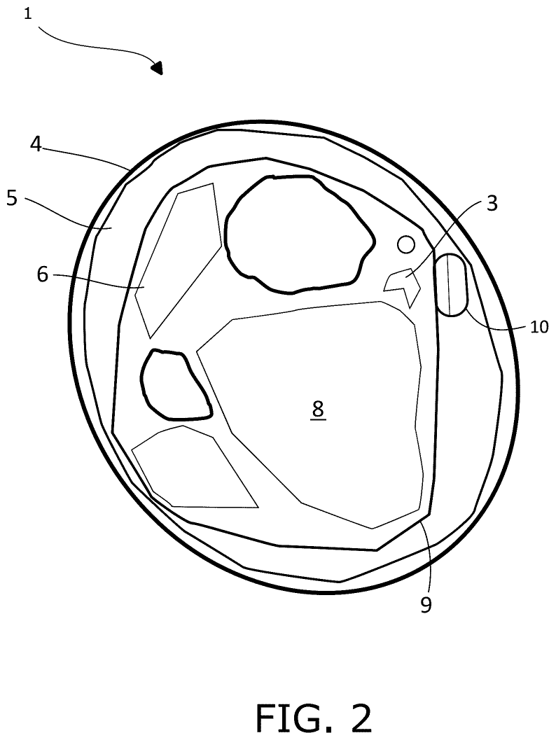

FIG. 2 shows a cross section of the patient's 1 leg implanted with the IPG 10 looking towards the body of the patient 1. The IPG 10 is configured to be implanted superficially, with the cathode positioned above the medial malleolus. The subcutaneous implantation provides for placing the IPG close to the surface of the skin 4 at a location just below the skin 4, rather than deeper within the body. The device 10 is preferably placed within the subcutaneous layer, which is the layer of fat and connective tissue directly beneath the skin. As shown in FIG. 2, the leg of the patient 1 includes the skin 4, the fat 5, the background tissue 6, the tibial nerve 3, the muscle 8, and the fascia 9. While the right leg is shown, the invention is not limited to one or the other legs; indeed, some patients may outlive their device and may eventually have a device in one leg, and then the other.

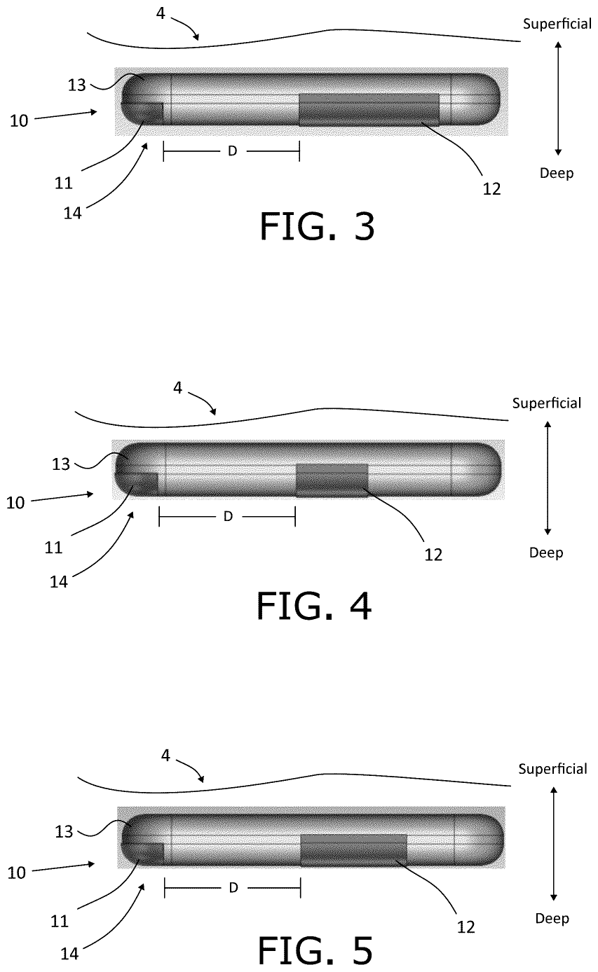

FIG. 3 shows one embodiment of the IPG 10 placed under the patient's skin 4. The IPG 10 includes the first electrode or cathode 11 and a second electrode or anode 12. The cathode 11 is positioned on the header portion 14, while the anode 12 (e.g., the return electrode) circumscribes a part of the housing 13. The remaining sections of the housing 13 are made of a non-conductive substrate, separating the cathode 11 and anode 12. In some embodiments, the substrate is formed entirely from a non-conductive material, such as a polymer insulator (e.g., a biocompatible epoxy) or ceramic. In this particular example, the housing 13 may be separately manufactured and the cathode 11 and the anode 12 subsequently attached or fitted into position. In other embodiments, the non-conductive material may be applied as a coating over an underlying conductive structure, with exposed conductive regions of the conductive structure defining the one or more of the cathode 11 and the anode 12. For example, the second electrode 12 may be an exposed portion of a conductive housing as shown at 13 in FIG. 1B, with remaining portions of the housing 13 covered by a non-conductive or insulating material.

An electrode offset or spacing may be referred to, and indicates, a distance between the anode and the cathode. In the illustrated embodiment, the IPG 10 includes an electrode offset of about 5 to about 20 millimeters, or about 10 mm. In other embodiments, distance ‘D’ may be manufactured at different offset lengths to accommodate the desired depth of tissue penetration and target nerve(s), which may range (typically) between 10-16 mm. Thus a range of devices and sizes may, optionally, be used or made available.

The IPG 10 includes circuitry for generating stimulation current or pulses that includes a current source configured to issue a constant current. Such an output can vary in voltage to maintain the required current output as tissue impedance varies. As higher voltage is demanded to provide the desired current output, more power is consumed by the current source circuitry to meet the current requirements. For example, a boost circuit may be used to boost a battery voltage (often around 3 to 3.5 volts for a single battery cell) to a higher voltage for therapy output purposes. Use of a boost circuit increases current consumption as some current is lost due to conversion.

Constant current output can be generated using a variety of known output circuitry topologies. A current mirror, or plural current mirrors, can be used to replicate a current that is controlled by a controller or microcontroller, for example. A feedback signal can be obtained from an output or return current by passing current that is directed to the output electrodes through a resistor; comparing the voltage drop across the resistor (which has a known value) to a voltage setpoint defined by a control circuit (such as a digital to analog conversion circuit and/or holding capacitor) allows the current delivered to be fixed at a constant level, for example and without limitation. Other designs can be used. While constant current therapy is noted, constant voltage or a voltage-controlled output can be used instead. In some examples, current-controlled or voltage-controlled outputs may be delivered as “square waves,” as is known in the art, though other examples may use other shapes, such as exponential, sinusoid, stepped, triangle, sawtooth, or other shapes if desired.

The electrode designs in the disclosed embodiments of the IPG provide for a device with both the higher power and voltage efficiency for the contemporary ITNS procedure. The electrode offset may vary to accommodate to fit the clinical goals of future ITNS procedures or otherwise modified to accommodate stimulation of other neuronal targets. Briefly referring to FIG. 2, the IPG 10 may be oriented vertically, at least when understood relative to a standing patient, such that the cathode is closer to the foot than the anode and the anode is closer to the knee than the cathode. This orientation may be, for example, more or less parallel to the direction of the tibia or fibula.

In FIG. 3, an illustrative set of dimensions for the implantable device 10 including the header 14, and the main body 13 may be 45 mm in length, 12 mm in width, and 6 mm in thickness and about 2.9 cc in volume. The cathode 11 may present a surface area of about 30 to 40 mm², and preferably about 34 mm². The anode 12 has a surface area of about 260 to 300 mm², and preferably about 285 mm². The anode 12 may extend along a longitudinal section of the main body of the IPG 10, wherein the anode 12 also extends circumferentially around the majority of the circumference of the main body. Other sizes and shapes may be used. The ends and sides of the device are as radiused, as shown, for enhanced patient comfort.

The device may be described as having first and second opposed planar surfaces—as shown in FIG. 3, these would be the upper and lower surfaces. The ends and sides of the device do not need to be flat. For example, in FIG. 3, only a small portion of the side surface (facing out on the page) is flat. The electrodes may extend into this flat surface, as shown at 12, or may not, as shown at 11. In an illustrative example, the region between the flat side surface and the larger planar surfaces on the top and bottom are connected by a continuous curved or radiused surface. The electrode 11 is thus defined with a portion on the planar lower surface (when implanted as shown in FIG. 3) and a curved portion which does not cross the device midline, as that term was defined previously. Electrode 12, in one example, extends to the planar side surface and thus extends beyond the midline. In an alternative, electrode 12 may stop short of the midline.

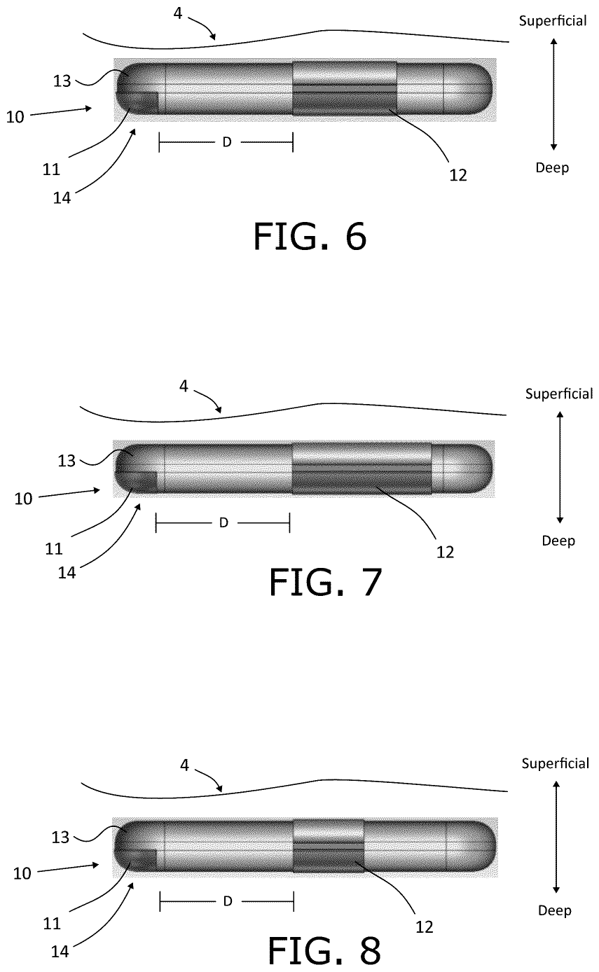

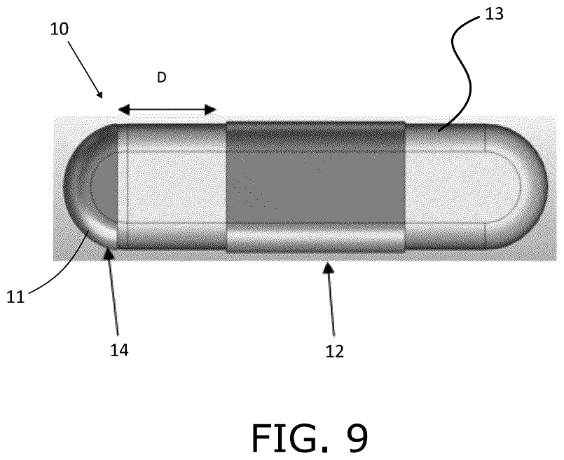

FIGS. 4-8 show variations of the IPG 10 placed under the patient's skin 4. The embodiments shown in FIGS. 3-5 illustrate configurations of the implantable device where both the cathode 11 and anode 12 are oriented to face inwards away from the skin 4, targeting the location of the tibial nerve. Alternatively, FIGS. 6-8 depict embodiments employing an annular anode design where the anode 12 extends around the entire circumference of the housing 13 of the implantable device 10. Across all embodiments, the offset ‘D’ may be about 6-16 mm, or about 8-12 mm, or about 10-14 mm. FIG. 9 shows the IPG 10 of the side facing the tibial nerve, with the preferrable offset ‘D’ of approximately 10 mm.

The various embodiments of the IPG shown in FIG. 4-8 are substantially the same design as the IPG 10 shown in FIG. 3, except for the configuration of the anode 12. The device shown in FIG. 4 has an anode with a surface area of 125 to 155 mm², and preferably about 142 mm². The anode 12 of the IPG 10 shown in FIG. 5 has a surface area of about 200 to 225 mm² and preferably about 213 mm². The anode 12 of the IPG 10 shown in FIG. 6 has a surface area of about 380 to 420 mm², and preferably about 407 mm². The anode 12 of the IPG 10 shown in FIG. 7 has a surface area of about 520 to 560 mm², and preferably about 543 mm². The anode 12 of the IPG 10 shown in FIG. 8 has a surface area of about 255 to 295 mm², and preferably about 272 mm². The device 10 shown in FIG. 9 is configured with the same anode 12 electrode as the IPG 10 in FIG. 3, with a surface area of about 260 to 300 mm², and preferably about 285 mm².

FIG. 4 shows an embodiment of an IPG 10 where the anode 12 is substantially smaller in surface area than the embodiments of the IPG 10 shown in FIGS. 3 and 5. The anode 12 of the IPG 10 shown in FIG. 5 is larger than the cathode 11 of the IPG 10 shown in FIG. 4, but smaller than the anode 12 in FIG. 3. In FIGS. 3-5, the anode 12 extends circumferentially around a portion of the housing 13 circumference such that a majority of the anode 12 faces the deep direction.

FIG. 6 shows an embodiment where the anode 12 is smaller in surface area than the embodiment shown in FIG. 7 but larger than the embodiment in FIG. 8. The anode 12 in FIG. 7 is larger than the embodiment shown in FIG. 6 and FIG. 8. In FIGS. 6-8, the anode 12 extends circumferentially around a majority of the housing 13 circumference such that the anode faces both the superficial and deep directions.

Each of the disclosed embodiments of implantable ITNS devices 10 provides significant advancement in volume of tissue activated (VTA) compared to conventional ITNS devices. The VTA is a volume of tissue in which the electrical field is of sufficient strength and gradient to likely cause a neuron to generate an action potential. VTA can be determined analytically, for example and without limitation, by use of finite element modeling and neuron simulation.

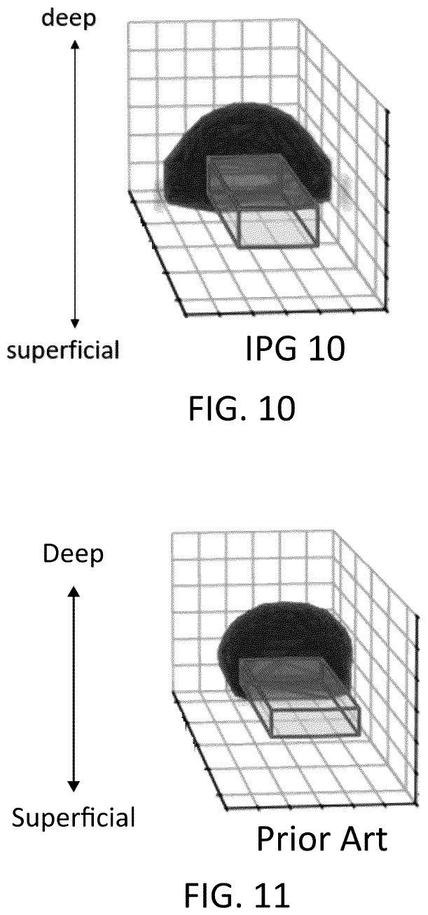

As depicted in FIG. 10, the VTA (shown in FIG. 10) generated by the device shown in FIG. 9 using a simplified homogenous tissue model is approximately 21% larger than the VTA (shown in FIG. 11) generated by a comparable conventional device. Notably, tibial nerve fibers exhibit a range of depths from the fascia, varying between 1.5 mm and 11.2 mm, with an average depth of 8 mm. As the distance between the tibial nerve and fascia increases, the energy required for neural recruitment also rises. The assumption underlying these measurements is that the ITNS device is positioned on or in proximity the fascia, without necessarily being attached or sutured to the fascia.

The expanded VTA provided by the disclosed devices (such as the device shown in FIG. 9) not only encompasses deep neural tissue, similar to existing devices, but also extends further than existing devices in the lateral direction. In the embodiment shown in FIG. 3, approximately 45 mm in length, 12 mm in width, and 6 mm in thickness with about 2.9 cc in volume, the VTA of the IPG 10 is approximately 4800 mm³. The VTA produced by the IPG 10 at a 3 mA stimulation level may, in some examples, be between about 4600 to 5000 mm³.

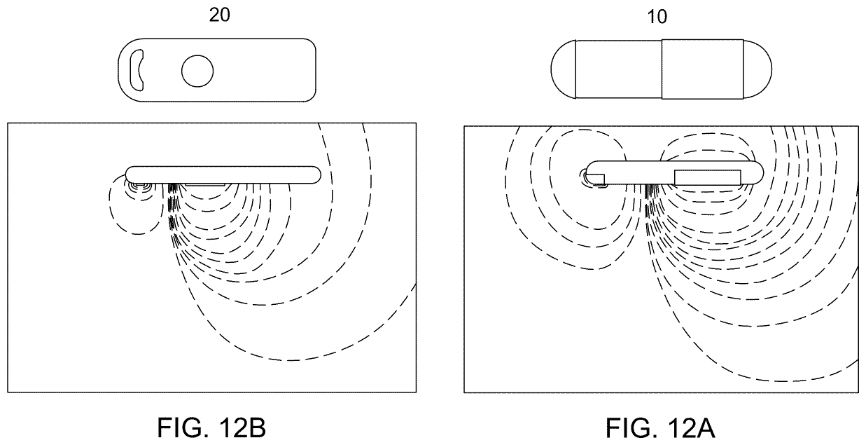

FIGS. 12A-12B illustrate a spatial voltage distribution of a comparable conventional device 20 and a spatial voltage distribution of the disclosed implantable device 10 during operation, respectively. The disclosed IPG exhibits a broader and deeper-reaching field compared to the conventional device. The voltage field generated by the conventional device exhibits a more localized and limited spatial extent, with higher concentrations near the electrode interface but diminished intensity at deeper tissue layers. In contrast, the disclosed device generates a field that extends farther from the electrode interface and penetrates deeper into the surrounding tissue, indicating improved spatial reach of the stimulation field; however, a larger spatial voltage distribution field does not necessarily correspond to a larger VTA.

Deeper voltage field penetration, brought about the design of the disclosed implantable device, may enable activation of neural targets located farther from the electrode, such as distal branches of the tibial nerve or other deep peripheral nerve fibers, without requiring an increase in stimulation intensity. This may also improve the efficacy of therapy in anatomically variable patients, for example, patients with higher amounts of fatty tissue or cases where the nerve targets are located farther apart.

The broadened voltage distribution is attributed, in part, to the electrode configuration of the disclosed IPG 10. The use of a larger or specially shaped anode, in conjunction with a strategically positioned cathode, results in a wider and more diffuse field distribution. The design promotes wider field coverage without compromising depth penetration, potentially enabling more effective engagement of neural structures located both in lateral and deep tissue.

The broader neural activation zone provided by the disclosed implantable devices and the associated novel electrode configuration, produces a larger therapeutic window, as shown in VTA results in FIG. 11. Consequently, the need for precise surgical placement is reduced resulting in a further simplification of the implantation procedure for the disclosed IPGs.

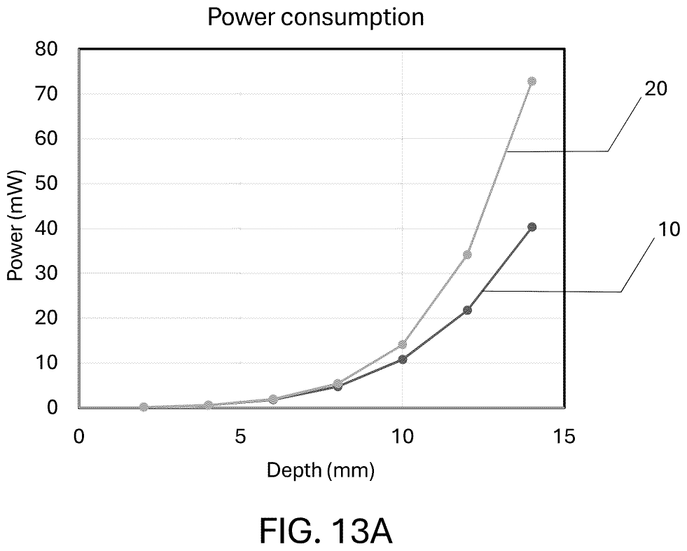

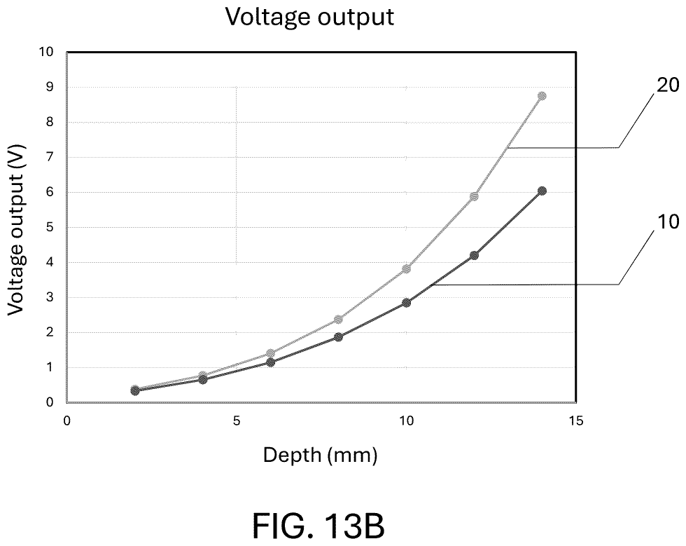

Furthermore, the design of the implantable stimulation device provides for improved power efficiency and effective tibial nerve stimulation. The disclosed device 10 provides for significantly lower impedance compared to conventional designs 20, primarily due to its substantially larger electrode surface area and unique electrode shape, as a combination results of the cathode and anode designs. The electrode configuration provides for lower power requirements (see FIG. 13A) and a lower voltage output (see FIG. 13B) required to elicit a neuronal response, a difference that becomes increasingly pronounced when targeting deeper neural tissue. Specifically, the disclosed embodiments exhibit an impedance of approximately 1 kΩ, while significantly reducing power consumption and thereby enabling a considerably longer battery life compared to existing technologies. Additional efficiencies may be gained by surface treatment or improvements such as laser texturing of electrodes, nitride coatings on titanium active surfaces, or IrO2 (Iridium Oxide) coatings on Pt—Ir active electrode surfaces.

The improvements contribute to maintaining, and in some cases widening, the therapeutic window, by reducing undesirable side effects, such as paresthesia, pain, muscle contractions, or general discomfort at or near the stimulation site. Compared to conventional devices, the design of the implantable stimulation device may decrease the risk of related side effects. Relatedly, the design of the device may result in a reduced-intensity stimulation programming, which may lead to greater long-term tolerability of the programming, with a reduced need for frequent reprogramming, less need for escalation of stimulation intensities, and fewer instances of therapy discontinuation due to general patient discomfort or other side effects.

Each of these non-limiting examples can stand on its own, or can be combined in various permutations or combinations with one or more of the other examples. The above detailed description includes references to the accompanying drawings, which form a part of the detailed description. The specification and drawings are, accordingly, to be regarded as illustrative rather than restrictive. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein. Other embodiments can be used, such as by one of ordinary skill in the art upon reviewing the above description. Further, the invention can be utilized in any number of environments and applications beyond those described herein without departing from the broader spirit and scope of the specification.

It will be recognized that the terms “comprising,” “including,” and “having,” as used herein, are specifically intended to be read as open-ended terms of art. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

In the event of inconsistent usages between this document and any documents so incorporated by reference, the usage in this document controls. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A leadless neurostimulation device configured to be implanted in a leg of a patient, the device comprising:
a header comprising a first electrode;
an elongated main body containing circuitry for generating stimulation current and a battery;
wherein the main body is connected to the header and includes a second electrode extending along a longitudinal section of the main body;
wherein the second electrode extends circumferentially around the majority of the main body.

2. The neurostimulation device of claim 1, wherein the second electrode extends around the entire circumference of the main body.

3. The neurostimulation device of claim 1, wherein the first electrode and the second electrode are on the same side of the main body.

4. The neurostimulation device of claim 1, wherein the neurostimulation device is configured to be implanted at a location about 2 cm to 8 cm above a medial malleolus of the patient.

5. The neurostimulation device of claim 1, wherein the neurostimulation device is configured to stimulate the tibial nerve.

6. The neurostimulation device of claim 1, wherein the first electrode includes a curved surface to direct stimulation current for both deep and lateral neural stimulation.

7. An implantable pulse generator for providing stimulation pulses to a patient, wherein the device is configured to be implanted in the leg of the patient, and wherein the device comprises:
a header portion;
a main body portion forming a case for stimulation circuitry and an energy storage device;
a first electrode positioned on an exterior of the header portion; and
a second electrode positioned on an exterior of the case.

8. The implantable pulse generator of claim 7, wherein the first electrode and the second electrode are on the same side of the implantable pulse generator.

9. The implantable pulse generator of claim 7, wherein the second electrode wraps completely around the case.

10. The implantable pulse generator of claim 7, wherein the first electrode includes a curved surface to direct stimulation current for both deep and lateral neural stimulation.

11. The implantable pulse generator of claim 7, wherein the first and second electrode are separated by a distance of about 10 mm.

12. The implantable pulse generator of claim 7, wherein the implantable pulse generator lacks separate leads, lead connectors, and external lead attachment points.

13. An implantable pulse generator for stimulation of the tibial nerve, comprising:
a housing including a sealed electronics compartment and a compartment for a battery;
a header portion connected to the housing,
a first electrode positioned on the header portion;
a second electrode extending along the housing and at least partially circumscribes the housing.

14. The implantable pulse generator of claim 13, wherein the second electrode completely circumscribes the housing.

15. The implantable pulse generator of claim 13, wherein the battery is non-rechargeable.

16. The implantable pulse generator of claim 13, wherein the first electrode includes a curved surface to direct stimulation current for both deep and lateral neural stimulation.

17. The implantable pulse generator of claim 13, wherein the first electrode is a cathode and the second electrode is an anode.

18. The implantable pulse generator of claim 13, wherein the first electrode and the second electrode are separated by a distance ranging from 10 mm to 16 mm.

19. The implantable pulse generator of claim 13, wherein the first electrode has a surface area of approximately 30 mm² to 40 mm².

20. The implantable pulse generator of claim 13, wherein the apparatus exhibits an impedance of approximately 1 kΩ during operation.
